(19) United States
(12) Patent Application Publication
(10) Pub. No.: US 2013/0131392 A1
(43) Pub. Date: May 23, 2013

(54) METHODS FOR PRODUCING ETHYL TERT-BUTYL ETHER

(71) Applicants: Jeannie Mee Blommel, Oregon, WI (US); Charles P. Luebke, Mount Prospect, IL (US)

(72) Inventors: Jeannie Mee Blommel, Oregon, WI (US); Charles P. Luebke, Mount Prospect, IL (US)

(73) Assignee: UOP LLC, Des Plaines, IL (US)

(21) Appl. No.: 13/300,192

(22) Filed: Nov. 18, 2011

(51) Int. Cl. C07C 41/42; C07C 43/04

(52) U.S. Cl. 568/697

(57) ABSTRACT

Embodiments of methods for producing ethyl tert-butyl ether (ETBE) are provided herein. A method for producing ETBE comprises the steps of contacting a C₄ olefin-rich stream that contains isobutene with a first catalyst in the presence of ethanol at etherification conditions effective to form a reaction effluent comprising ETBE and residual ethanol. The reaction effluent is combined with a C₅ paraffin-rich stream that contains isopentane to form a combined stream. The combined stream is fractionated at distillation conditions effective to produce an ETBE-rich product stream. Fractionating the combined stream comprises forming a light boiling azeotrope of ethanol with isopentane to remove at least a portion of the residual ethanol from the combined stream.

### TECHNICAL FIELD

The present invention relates generally to methods for producing ethers from alcohols and olefins, and more particularly relates to methods for producing ethyl tert-butyl ether from ethanol and a C₄ olefin-rich stream that contains isobutene.

### BACKGROUND

Ethyl tert-butyl ether (ETBE) is commonly used as an oxygenate gasoline additive in the production of gasoline from crude oil, for example, to increase the octane rating of the gasoline. ETBE offers improved air quality benefits over other oxygenate gasoline additives such as ethanol. Unlike ethanol, ETBE does not promote evaporation of gasoline, which is one of the causes of smog, and does not readily absorb moisture from the atmosphere.

ETBE is typically produced from the catalytic etherification of ethanol with C₄ branched olefins. In one example, a feed stream containing various C₄⁻ hydrocarbons including some C₄ branched olefins such as isobutene (e.g. up to about 25 weight percent (wt. %)), is passed over a catalyst together with an excess of ethanol to form a reaction effluent that comprises ETBE, unreacted C₄⁻ hydrocarbons (e.g. C₄⁻ normal olefins and/or paraffins), unreacted ethanol, and an oxygenate byproduct. As used herein, Cₓ means hydrocarbon molecules that have “X” number of carbon atoms, Cₓ⁺ means hydrocarbon molecules that have “X” and/or more than “X” number of carbon atoms, and Cₓ⁻ means hydrocarbon molecules that have “X” and/or less than “X” number of carbon atoms. As used herein, the term “rich” can mean an amount of at least generally about 50%, such as about 70%, by mole, of a compound or class of compounds in a stream. To form a product stream that is rich in ETBE, much of the unreacted components and byproducts are separated from the reaction effluent. The reaction effluent can be fractionated, for example, by distillation to remove a significant portion of the oxygenate byproduct, the unreacted C₄⁻ hydrocarbons, and the unreacted ethanol. During distillation, ethanol forms a weak azeotrope with the unreacted C₄ hydrocarbons (e.g. the composition of boiling azeotropic mixture has a relatively low concentration of ethanol with a relatively high concentration of C₄ hydrocarbons) and is slowly removed from the reaction effluent with the C₄ hydrocarbons in an azeotropic vapor phase.

Recently, some ETBE producers have been evaluating the feasibility of using feed streams with higher concentrations of isobutene, e.g., about 40 to 45 wt. %, to improve their production yields. Isobutene has a relatively high conversion rate and selectivity for reacting with ethanol to produce ETBE and therefore, feed streams that contain higher concentrations of isobutene can effectively produce reaction effluents with increased amounts of ETBE. However, producing more ETBE means that more C₄ hydrocarbons (e.g. isobutene) from the feed stream are consumed, leaving less unreacted C₄ hydrocarbons in the reaction effluent for removing the unreacted ethanol via distillation or the like. Unfortunately, this makes the task of fractionating the reaction effluent to produce an ETBE-rich product stream more challenging.

Accordingly, it is desirable to provide methods for producing ETBE from the catalytic etherification of ethanol with a feed stream that contains relatively high amounts of isobutene. Moreover, it is desirable to provide methods for producing ETBE including removing unreacted ethanol from the reaction effluent to form an ETBE-rich product stream. Furthermore, other desirable features and characteristics of the present invention will become apparent from the subsequent detailed description and the appended claims, taken in conjunction with the accompanying drawings and this background.

### BRIEF SUMMARY

Methods for producing ethyl tert-butyl ether (ETBE) are provided herein. In accordance with an exemplary embodiment, a method for producing ETBE comprises the steps of contacting a C₄ olefin-rich stream that contains isobutene with a first catalyst in the presence of ethanol at etherification conditions effective to form a reaction effluent comprising ETBE and residual ethanol. The reaction effluent is combined with a C₅ paraffin-rich stream that contains isopentane to form a combined stream. The combined stream is fractionated at distillation conditions effective to produce an ETBE-rich product stream. Fractionating the combined stream comprises forming a light boiling azeotrope of ethanol with isopentane to remove at least a portion of the residual ethanol from the combined stream.

In accordance with another exemplary embodiment, a method for producing ETBE is provided. The method comprises the steps of introducing a stoichiometric excess of ethanol and a C₄ olefin-rich stream that contains isobutene to a reaction zone. The reaction zone contains a first catalyst and is operating at etherification conditions effective to form a reaction effluent comprising ETBE, an oxygenate byproduct, residual isobutene, and residual ethanol. The reaction effluent and a C₅ paraffin-rich stream that contains isopentane are introduced to a reactive distillation zone that contains a second catalyst. The reactive distillation zone is operating at reactive distillation conditions effective to react at least a portion of the residual isobutene with a first portion of the residual ethanol to produce additional ETBE and to remove the oxygenate byproduct and a second portion of the residual ethanol to produce an ETBE-rich product stream. Removing the second portion of the residual ethanol includes forming a light boiling azeotrope of ethanol with isopentane to remove the second portion of the residual ethanol together with isopentane.

In accordance with another exemplary embodiment, a method for producing ETBE is provided. The method comprises the steps of feeding ethanol and a C₄ olefin-rich stream that contains isobutene and propane to a reaction zone. The reaction zone contains a first catalyst and is operating at etherification conditions effective to form a reaction effluent comprising ETBE, propane, an oxygenate byproduct, residual isobutene, and residual ethanol. The reaction effluent is combined with a C₅ paraffin-rich stream that contains isopentane to form a combined stream. The combined stream is fed to a reactive distillation zone. The reactive distillation zone contains a second catalyst and is operating at reactive distillation conditions effective to produce an ETBE-rich product stream. Feeding the combined stream comprises reacting at least a portion of the residual isobutene with a first portion of the residual ethanol to produce additional ETBE. The oxygenate byproduct, C₅⁻ hydrocarbons, and a second portion of the residual ethanol are removed to produce the ETBE-rich product stream and an intermediate effluent stream. The intermediate effluent stream comprises the second portion of the residual ethanol, the oxygenate byproduct, and C₅⁻ hydrocarbons. Removing the second portion of the residual ethanol includes forming a light boiling azeotrope of ethanol with isopentane to remove the second portion of the residual ethanol together with isopentane. The intermediate effluent stream is separated into an ethanol-rich stream, a propane-rich stream, a C₅ hydrocarbons-oxygenate byproduct stream, and an oxygenate-depleted C₄ hydrocarbons-containing raffinate.

### BRIEF DESCRIPTION OF THE DRAWINGS

The present invention will hereinafter be described in conjunction with the following drawing figures, wherein like numerals denote like elements, and wherein:

FIG. 1 schematically illustrates an apparatus for producing ethyl tert-butyl ether (ETBE) in accordance with an exemplary embodiment.

### DETAILED DESCRIPTION

The following Detailed Description is merely exemplary in nature and is not intended to limit the invention or the application and uses of the invention. Furthermore, there is no intention to be bound by any theory presented in the preceding background or the following detailed description.

Various embodiments contemplated herein relate to methods for producing ethyl tert-butyl ether (ETBE). Unlike the prior art, the exemplary embodiments taught herein include contacting a C₄ olefin-rich stream that contains isobutene with a catalyst in the presence of ethanol at etherification conditions effective to form a reaction effluent comprising ETBE and residual ethanol (e.g. unreacted ethanol). In an exemplary embodiment, the C₄ olefin-rich stream comprises isobutene that is present in an amount of about 40 weight percent (wt. %) or greater.

The reaction effluent is combined with a C₅ paraffin-rich stream that contains isopentane to form a combined stream. The combined stream is fractionated at distillation conditions effective to produce an ETBE-rich product stream. During fractionation, a light boiling azeotrope of ethanol with isopentane is formed such that the residual ethanol is steadily removed from the combined stream with the isopentane in an azeotropic vapor phase. As used herein, the phrase “light boiling azeotrope” means that the boiling point of the azeotrope is less than its primary constituent(s) (e.g. also referred to as a positive azeotrope). Thus, a light boiling azeotrope of ethanol with isopentane means that the boiling point of the azeotrope at a particular pressure is less than the boiling point of pure ethanol at the particular pressure or that the boiling point of the azeotrope is less than the boiling points of pure ethanol and pure isopentane at the particular pressure. The inventors have found that the residual ethanol can be removed efficiently with isopentane even if the reaction effluent has relatively low amounts of unreacted C₄ hydrocarbons present because the concentration of ethanol in the light boiling azeotrope with isopentane is relatively high compared to the concentration of ethanol in the weak azeotrope that is otherwise formed with C₄ hydrocarbons. Therefore, the residual ethanol can be effectively removed from a reaction effluent, which has been formed for example from ethanol and relatively high amounts of isobutene, to produce an ETBE-rich product stream.

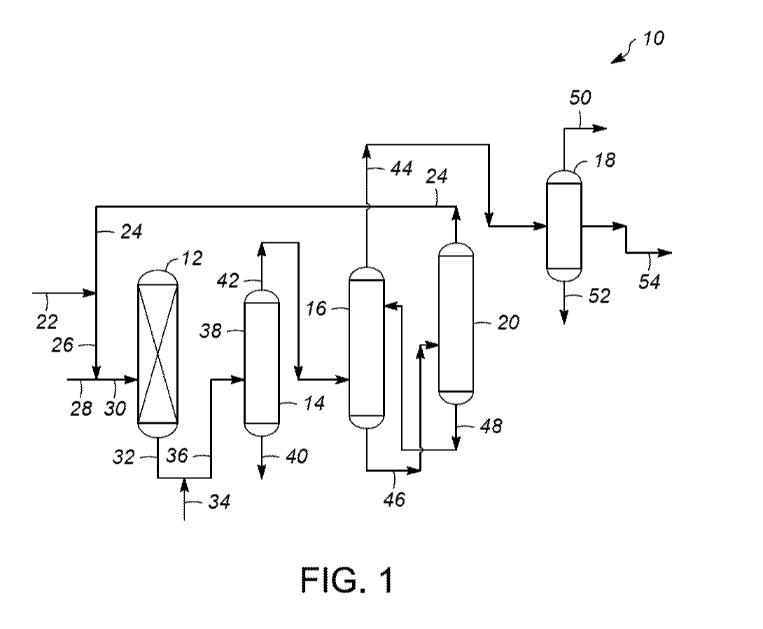

Referring to FIG. 1, a block diagram of an apparatus 10 for producing ETBE in accordance with an exemplary embodiment is provided. As illustrated, the apparatus 10 comprises a reaction zone 12, a distillation zone 14, a water wash extraction zone 16, a hydrocarbon fractionation zone 18, and an alcohol-water fractionation zone 20. As used herein, the term “zone” can refer to an area including one or more equipment items and/or one or more sub-zones. Equipment items can include one or more reactors or reactor vessels, fractionation columns, heaters, exchangers, pipes, pumps, compressors, controllers, dryers, vessels, and the like.

A fresh ethanol-rich stream 22 is combined with a recycle ethanol-rich stream 24 as will be discussed in further detail below to form an ethanol-rich feed stream 26. The ethanol-rich feed stream 26 is combined with a C₄ olefin-rich feed stream 28 to form a combined feed stream 30. The C₄ olefin-rich feed stream 28 is rich in C₄ olefins, such as 1-butene, cis- and/or trans-2-butene, and isobutene. In an exemplary embodiment, the C₄ olefin feed stream 28 comprises isobutene present in an amount of about 40 wt. % or greater, such as about 40 to about 60 wt. %. The C₄ olefin feed stream 28 may also contain other C₄⁻ olefins and/or paraffins, such as propane. In an exemplary embodiment, the combined feed stream 30 has a stoichiometric excess of ethanol to isobutene. In one example, the combined feed stream 30 has a molar ratio of ethanol to isobutene of from about 1.02 to about 1.2.

The combined feed stream 30 is introduced to the reaction zone 12. The reaction zone 12 may be configured comprising one or more reactors that contain a catalyst for the catalytic etherification of ethanol with C₄ branched olefins. In an exemplary embodiment, the catalyst is a standard or high activity ion-exchange resin that may be strongly acidic, such as an ion-exchange resin of macroreticulated sulfonic type. Other suitable catalyst known to those skilled in the art for the catalytic etherification of ethanol with C₄ branched olefins may also be used.

In an exemplary embodiment, the combined feed stream 30 contacts the catalyst while the reaction zone 12 is operating at etherification conditions such that the etherification reaction is carried out in the liquid phase with a molar excess of ethanol to isobutene so as to shift the equilibrium to the production of ether, such as ETBE, and to form a reaction effluent 32. In one example, the etherification conditions include a temperature of from about 30 to about 75° C. In another example, the etherification conditions include a pressure of from about 0.5 to about 2 MPa. In an exemplary embodiment, about 95 mole % or greater of the isobutene is converted to ETBE, such as from about 95 to about 97 mole %. Other products can be formed during the etherification reaction, such as oxygenate byproducts formed from the reaction of ethanol with ethanol, for example diethyl ether (DEE). In one example, the reaction effluent 32 comprises ETBE and residual ethanol, and can further comprise an oxygenate byproduct such as DEE, residual isobutene, and/or other C₄⁻ hydrocarbons such as propane.

The reaction effluent 32 is removed from the reaction zone 12 and is combined with a C₅ paraffin-rich stream 34 to form a combined stream 36. The C₅ paraffin-rich stream 34 is rich in C₅ paraffins such as isopentane and may contain other C₅⁻ hydrocarbons. In an exemplary embodiment, the reaction effluent 32 is combined with the C₅ paraffin-rich stream 34 at a weight ratio of the residual ethanol to isopentane of from about 1:10 to about 2:5.

The combined stream 36 is introduced to the distillation zone 14. The distillation zone 14 is operating at distillation conditions effective to fractionate the combined stream 36 to produce an ETBE-rich product stream 40 and an intermediate effluent stream 42 that comprises isopentane and at least portions of the residual ethanol and isobutene, and further can also comprise the oxygenate byproduct such as DEE and/or other C₅⁻ hydrocarbons such as propane. In an exemplary embodiment, the ETBE-rich product stream 40 comprises ETBE that is present in an amount of about 95 wt. % or greater, such as about 97 wt. % or greater, such as about 99 wt. % or greater.

In an exemplary embodiment, the distillation zone 14 is configured as a reactive distillation zone 38. The reactive distillation zone 38 is operating at reactive distillation conditions effective to further react and fractionate the combined stream 36. In one example, the reactive distillation zone 38 contains a catalyst that is similar to the catalyst contained in the reaction zone 12, such as a standard or high activity ion-exchange resin that may be strongly acidic. Other suitable catalyst known to those skilled in the art for the catalytic etherification of ethanol with C₄ branched olefins may also be used. Prior to or during fractionation, the combined stream 36 contacts the catalyst and at least a portion of the residual ethanol and at least a portion of the residual isobutene react to form additional ETBE. In one example, an additional amount of about 1 to about 4 mole % of the original isobutene contained in the combined feed stream 30 is converted in the reactive distillation zone 38 to ETBE for a total conversion of isobutene to ETBE of about 96 to about 99.5 mole %.

In an exemplary embodiment, the combined stream 36 is fractionated at the distillation conditions in the distillation zone 14, which can be configured as the reactive distillation zone 38 at reactive distillation conditions as discussed above or otherwise, such that a light boiling azeotrope comprising isopentane and a remaining portion of the residual ethanol is formed. It has been found that concentration of ethanol present in the light boiling azeotrope formed with isopentane is dependent upon the operating pressure of the distillation zone 14 but is relatively high for a range of operating pressures and is generally about 5 times or greater than the concentration of ethanol present in the weak azeotrope formed with C₄ hydrocarbons. Ethanol is steadily removed from the combined stream 36 in an azeotropic vapor phase with the isopentane. Additionally, the oxygenate byproduct such as DEE and/or other C₅⁻ hydrocarbons such as propane are removed from the combined stream 36 during distillation along with the azeotropic vapor phase to form the intermediate effluent stream 42. In an exemplary embodiment, the distillation conditions (e.g., reactive distillation conditions) include a pressure of from about 550 to about 870 kPa gauge and a temperature of from about 50 to about 75° C.

The ETBE-rich product stream 40 is removed from the distillation zone 14, e.g., as a bottom stream, and the intermediate effluent stream 42 is removed from the distillation zone 14, e.g., as an overhead stream. As illustrated, the intermediate effluent stream 42 is passed along to the water wash extraction zone 16. The water wash extraction zone 16 is configured for contacting the intermediate effluent stream 42, e.g., flowing upwardly through the water wash extraction zone 16, with water that is flowing countercurrent to the intermediate effluent stream 42, e.g., flowing downwardly through the water wash extraction zone 16. The water wash extraction zone 16 is operating at liquid-liquid extraction conditions effective to remove the residual ethanol and form an ethanol-depleted intermediate stream 44 and an ethanol-water rich stream 46. In an exemplary embodiment, the liquid-liquid extraction conditions include a temperature of from about 25 to about 50° C. and a pressure high enough to maintain the intermediate effluent stream 42, the ethanol-depleted intermediate stream 44, the ethanol-water rich stream 46, and water in a liquid phase.

The ethanol-water rich stream 46 is removed from the water wash extraction zone 16 and is passed along to the alcohol-water fractionation zone 20. In an exemplary embodiment, the alcohol-water fractionation zone 20 is operating at fractionation conditions effective to separate the ethanol-water rich stream 46 into the recycled ethanol-rich stream 24 that is combined with the fresh ethanol-rich stream 22, and a water-rich stream 48 that is passed along to the water wash extraction zone 16.

The ethanol-depleted intermediate stream 44 is removed from the water wash extraction zone 16 and is passed along to the hydrocarbon fractionation zone 18. In an exemplary embodiment, the ethanol-depleted intermediate stream comprises an oxygenate byproduct and C₅⁻ hydrocarbons. The hydrocarbon fractionation zone 18 is operating at fractionation conditions effective to separate the ethanol-depleted intermediate stream 44 into a propane-rich stream 50, a C₅ hydrocarbons-oxygenate byproduct stream 52, and an oxygenate-depleted C₄ hydrocarbons-containing raffinate 54.

Accordingly, methods for producing ETBE have been described. The various embodiments contemplated herein include contacting a C₄ olefin-rich stream that contains isobutene with a catalyst in the presence of ethanol at etherification conditions effective to form a reaction effluent comprising ETBE and residual ethanol. The reaction effluent is combined with a C₅ paraffin-rich stream that contains isopentane to form a combined stream. The combined stream is fractionated at distillation conditions effective to produce an ETBE-rich product stream. During fractionation, a light boiling azeotrope of ethanol with isopentane is formed such that the residual ethanol is steadily removed from the combined stream with the isopentane in an azeotropic vapor phase. The residual ethanol can be removed efficiently with isopentane even if the reaction effluent has relatively low amounts of unreacted C₄ hydrocarbons present because the concentration of ethanol in the light boiling azeotrope formed with isopentane is relatively high compared to the concentration of ethanol in the weak azeotrope that is otherwise formed with C₄ hydrocarbons. Therefore, the residual ethanol can be effectively removed from a reaction effluent, which has been formed for example from ethanol and relatively high amounts of isobutene, to produce an ETBE-rich product stream.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for producing ethyl tert-butyl ether (ETBE), the method comprising the steps of:
contacting a C₄ olefin-rich stream that contains isobutene with a first catalyst in the presence of ethanol at etherification conditions effective to form a reaction effluent comprising ETBE and residual ethanol;
combining the reaction effluent with a C₅ paraffin-rich stream that contains isopentane to form a combined stream; and
fractionating the combined stream at distillation conditions effective to produce an ETBE-rich product stream, wherein fractionating the combined stream comprises forming a light boiling azeotrope of ethanol with isopentane to remove at least a portion of the residual ethanol from the combined stream.

2. The method of claim 1, wherein the step of contacting the C₄ olefin-rich stream comprises contacting the C₄ olefin-rich stream that comprises isobutene present in an amount of about 40 wt. % or greater of the C₄ olefin-rich stream.

3. The method of claim 1, wherein the step of contacting the C₄ olefin-rich stream comprises contacting the C₄ olefin-rich stream that comprises isobutene present in an amount of about 40 to about 60 wt. % of the C₄ olefin-rich stream.

4. The method of claim 1, wherein the step of contacting the C₄ olefin-rich stream comprises reacting isobutene with ethanol at a molar ratio of ethanol to isobutene of from about 1.02 to about 1.2.

5. The method of claim 1, wherein the step of contacting the C₄ olefin-rich stream comprises contacting the C₄ olefin-rich stream at the etherification conditions that include a temperature of from about 30 to about 75° C.

6. The method of claim 1, wherein the step of combining the reaction effluent comprises combining the reaction effluent with the C₅ paraffin-rich stream at a weight ratio of the residual ethanol to isopentane of from about 1:10 to about 2:5.

7. The method of claim 1, wherein the step of fractionating the combined stream comprises fractionating the combined stream at the distillation conditions that include a pressure of from about 550 to about 827 kPa gauge.

8. The method of claim 1, wherein the step of fractionating the combined stream comprises fractionating the combined stream at the distillation conditions that include a temperature of from about 50 to about 75° C.

9. The method of claim 1, wherein the step of fractionating the combined stream comprises forming the light boiling azeotrope having a boiling temperature at the distillation conditions that is less than a boiling point for pure ethanol at the distillation conditions.

10. The method of claim 1, wherein the step of contacting the C₄ olefin-rich stream comprises forming the reaction effluent that comprises ETBE, the residual ethanol, and residual isobutene, and the method further comprises the step of:
contacting the combined stream with a second catalyst to react a first portion of the residual isobutene with a second portion of the residual ethanol to form additional ETBE prior to or during the step of fractionating the combined stream.

11. The method of claim 1, wherein the step of contacting the C₄ olefin-rich stream comprises forming the reaction effluent that comprises ETBE, propane, the residual ethanol, and an oxygenate byproduct, wherein the step of fractionating the combined stream comprises forming an intermediate effluent stream that comprises the portion of the residual ethanol, the oxygenate byproduct, and C₅− hydrocarbons, and wherein the method further comprises the step of:
contacting the intermediate effluent stream with water at liquid-liquid extraction conditions effective to remove the residual ethanol and to form an ethanol-depleted intermediate stream that comprises the oxygenate byproduct and the C₅⁻ hydrocarbons.

12. The method of claim 11, further comprising the step of:
separating the ethanol-depleted intermediate stream into a propane-rich stream, a C₅ hydrocarbons-oxygenate byproduct stream, and an oxygenate-depleted C₄ hydrocarbons-containing raffinate.

13. The method of claim 11, wherein the step of contacting the intermediate effluent stream comprises forming an ethanol-water rich stream, and wherein the method further comprises the step of:
separating the ethanol-water rich stream into an ethanol-rich stream and a water-rich stream.

14. A method for producing ethyl tert-butyl ether (ETBE), the method comprising the steps of:
introducing a stoichiometric excess of ethanol and a C₄ olefin-rich stream that contains isobutene to a reaction zone that contains a first catalyst and that is operating at etherification conditions effective to form a reaction effluent comprising ETBE, an oxygenate byproduct, residual isobutene, and residual ethanol; and
introducing the reaction effluent and a C₅ paraffin-rich stream that contains isopentane to a reactive distillation zone that contains a second catalyst, wherein the reactive distillation zone is operating at reactive distillation conditions effective to react at least a portion of the residual isobutene with a first portion of the residual ethanol to produce additional ETBE and to remove the oxygenate byproduct and a second portion of the residual ethanol to produce an ETBE-rich product stream, and wherein removing the second portion of the residual ethanol includes forming a light boiling azeotrope of ethanol with isopentane to remove the second portion of the residual ethanol together with isopentane.

15. The method of claim 14, wherein the step of introducing the stoichiometric excess of ethanol comprises forming the reaction effluent that comprises ETBE, propane, the oxygenate byproduct, the residual isobutene, and the residual ethanol, wherein the step of introducing the reaction effluent comprises forming an intermediate effluent stream that comprises the second portion of the residual ethanol, the oxygenate byproduct, and C₅− hydrocarbons, and wherein the method further comprises the step of:
introducing the intermediate effluent stream to a water wash extraction zone operating at liquid-liquid extraction conditions effective to remove the second portion of the residual ethanol and to form an ethanol-depleted intermediate stream that comprises the oxygenate byproduct and the C₅⁻ hydrocarbons.

16. The method of claim 15, further comprising the step of:
introducing the ethanol-depleted intermediate stream to a hydrocarbon fractionation zone operating at fractionation conditions effective to separate the ethanol-depleted intermediate stream into a propane-rich stream, a C₅ hydrocarbons-oxygenate byproduct stream, and an oxygenate-depleted C₄ hydrocarbons-containing raffinate.

17. The method of claim 15, wherein the step of introducing the intermediate effluent stream comprises forming an ethanol-water rich stream, and wherein the method further comprises the step of:
introducing the ethanol-water rich stream to an alcohol-water fractionation zone operating at fractionation conditions effective to separate the ethanol-water rich stream into an ethanol-rich stream and a water-rich stream.

18. A method for producing ethyl tert-butyl ether (ETBE), the method comprising the steps of:
feeding ethanol and a C₄ olefin-rich stream that contains isobutene and propane to a reaction zone that contains a first catalyst and that is operating at etherification conditions effective to form a reaction effluent comprising ETBE, propane, an oxygenate byproduct, residual isobutene, and residual ethanol;
combining the reaction effluent with a C₅ paraffin-rich stream that contains isopentane to form a combined stream;
feeding the combined stream to a reactive distillation zone that contains a second catalyst and that is operating at reactive distillation conditions effective to produce an ETBE-rich product stream, wherein feeding the combined stream comprises:
reacting at least a portion of the residual isobutene with a first portion of the residual ethanol to produce additional ETBE; and
removing the oxygenate byproduct, C₅⁻ hydrocarbons, and a second portion of the residual ethanol to produce the ETBE-rich product stream and an intermediate effluent stream that comprises the second portion of the residual ethanol, the oxygenate byproduct, and C₅⁻ hydrocarbons, and wherein removing the second portion of the residual ethanol includes forming a light boiling azeotrope of ethanol with isopentane to remove the second portion of the residual ethanol together with isopentane; and
separating the intermediate effluent stream into an ethanol-rich stream, a propane-rich stream, a C₅ hydrocarbons-oxygenate byproduct stream, and an oxygenate-depleted C₄ hydrocarbons-containing raffinate.

19. The method of claim 18, wherein the step of feeding ethanol comprises feeding ethanol and the C₄ olefin-rich stream to the reaction zone at a molar ratio of ethanol to isobutene of from about 1.02 to about 1.2.

20. The method of claim 18, wherein the step of combining the reaction effluent comprises combining the reaction effluent with the C₅ paraffin-rich stream at a weight ratio of the residual ethanol to isopentane of from about 1:10 to about 2:5.
